(19) United States
(12) Patent Application Publication
(10) Pub. No.: US 2023/0135782 A1
(43) Pub. Date: May 4, 2023

(54) DISABILITY LEVEL AUTOMATIC JUDGMENT DEVICE AND A DISABILITY LEVEL AUTOMATIC JUDGMENT METHOD

(71) Applicant: INSTITUTE FOR INFORMATION INDUSTRY, TAIPEI (TW)

(72) Inventors: Tai-Ta KUO, Taipei (TW); Yu-Chuan YANG, Taipei (TW); Jia Wei KAO, Taipei (TW); Fu-Jheng JHENG, Taipei (TW); Yi Hsiu LEE, Taipei (TW); Ping-I CHEN, Taipei (TW)

(21) Appl. No.: 17/456,588

(22) Filed: Nov. 25, 2021

(30) Foreign Application Priority Data
Nov. 3, 2021 (TW) 110141034

(51) Int. Cl. G06Q 40/08; G06N 5/02; G16H 10/60

(52) U.S. Cl. CPC G06Q 40/08; G06N 5/02; G16H 10/60

(57) ABSTRACT

A disability level automatic judgment device is disclosed. The disability level automatic judgment device includes a processor and a memory. The processor is configured to create a diagnosis information graph according to a diagnosis content, to compare the diagnosis information graph and a standard disability graph, so as to determine a first disability level, and to generate a judgment result according to the first disability level. The memory is coupled to the processor, and the memory is configured to store the standard disability graph.

### CROSS-REFERENCE TO RELATED APPLICATION

This application claims the priority benefit of TAIWAN Application serial no. 110141034, filed Nov. 3, 2021, the full disclosure of which is incorporated herein by reference.

### BACKGROUND

### Field of Invention

The invention relates to a disability level automatic judgment device and a disability level automatic judgment method. More particularly, the invention relates to a disability level automatic judgment device and a disability level automatic judgment method for performing a disability level determination by creating the knowledge graph.

### Description of Related Art

The disability determination of general medical insurance claims involves complicated medical knowledge and the inconsistency of diagnostic certificates issued by medical institutions. Therefore, it must rely on the judgment of professionals before entering the insurance claims system, which requires a lot of manpower and the processing speed is slow. It will increase the labor cost of insurance companies and slow down the speed of claims settlement.

The existing claims system has proposed automatic judgment of the degree of human injury or disability. It creates specific models and uses big data for training to generate classification and corresponding results, or keywords and preset claims rules are used for determination. However, the disadvantage is that the accuracy is not high and it is difficult to replace the professionals.

### SUMMARY

An aspect of this disclosure is to provide a disability level automatic judgment device. The disability level automatic judgment device includes a processor and a memory. The processor is configured to create a diagnosis information graph according to a diagnosis content, to compare the diagnosis information graph and a standard disability graph, so as to determine a first disability level, and to generate a judgment result according to the first disability level. The memory is coupled to the processor, and the memory is configured to store the standard disability graph.

Another aspect of this disclosure is to provide a disability level automatic judgment method. The disability level automatic judgment method includes the following operations: storing a standard disability graph by a memory; creating a diagnosis information graph according to a diagnosis content by a processor; comparing the diagnosis information graph and the standard disability graph, so as to determine a first disability level by the processor; and generating a judgment result according to the first disability level by the processor.

### BRIEF DESCRIPTION OF THE DRAWINGS

Aspects of the present disclosure are best understood from the following detailed description when read with the accompanying figures. It is noted that, according to the standard practice in the industry, various features are not drawn to scale. In fact, the dimensions of the various features may be arbitrarily increased or reduced for clarity of discussion.

FIG. 1 is a schematic diagram illustrating a disability level automatic judgment device according to some embodiments of the present disclosure.

FIG. 2 is a flowchart illustrating a disability level automatic judgment method according to some embodiments of the present disclosure.

FIG. 3 is a schematic diagram illustrating a standard disability graph according to some embodiments of the present disclosure.

FIG. 4 is a schematic diagram illustrating a diagnosis according to some embodiments of the present disclosure.

FIG. 5 is a flowchart illustrating one of the operations illustrated in FIG. 2 according to some embodiments of the present disclosure.

FIG. 6 is a schematic diagram illustrating a diagnosis information graph according to some embodiments of the present disclosure.

FIG. 7 is a schematic diagram illustrating a comparison result according to some embodiments of the present disclosure.

FIG. 8 is a schematic diagram illustrating a judgment result according to some embodiments of the present disclosure.

### DETAILED DESCRIPTION

The following disclosure provides many different embodiments, or examples, for implementing different features of the invention. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention.

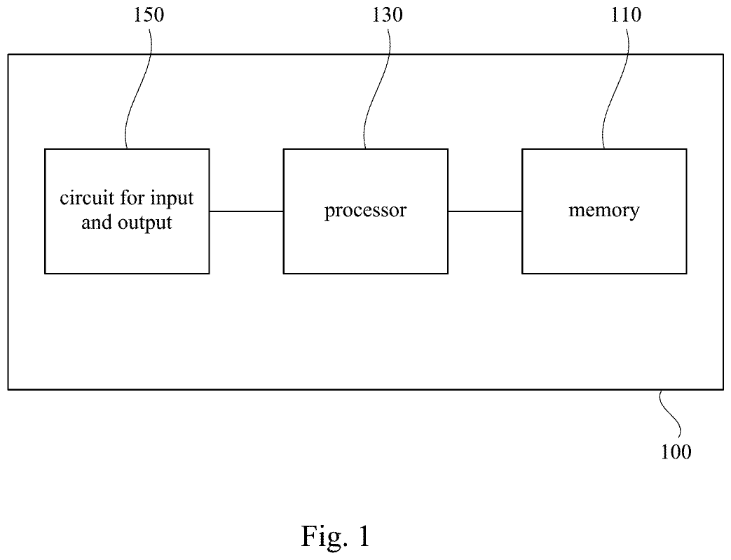

Reference is made to FIG. 1. FIG. 1 is a schematic diagram illustrating a disability level automatic judgment device 100 according to some embodiments of the present disclosure. In some embodiments, the disability level automatic judgment device 100 includes the memory 110 and the processor 130. The memory 110 couples to the processor 130. In some embodiments, the memory 110 further includes the circuit for input and output 150. The circuit for input and output 150 couples to the processor 130.

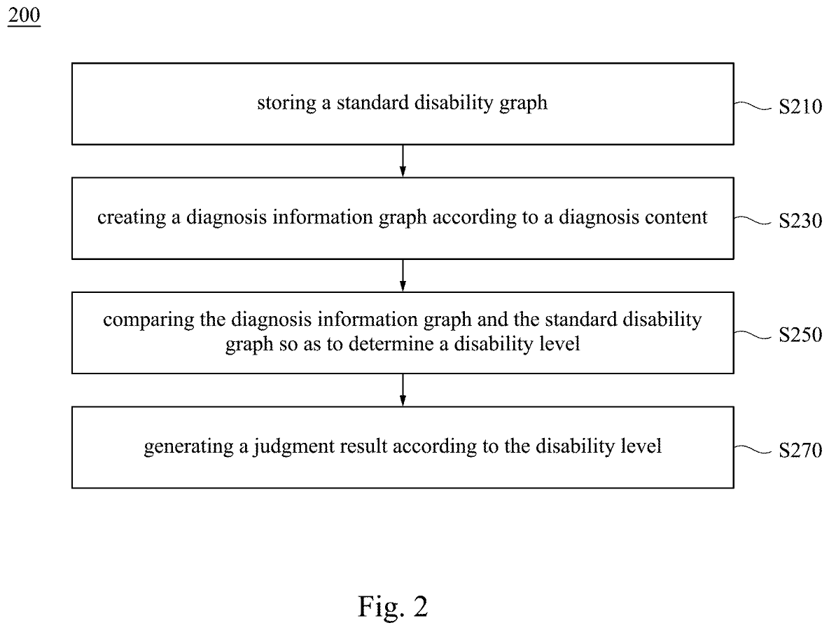

The disability level automatic judgment device 100 shown in FIG. 1 is only for illustrative purposes, and the embodiments of the present disclosure are not limited thereto. Regarding the operation method of disability level automatic judgment device 100, reference is made to FIG. 2 below.

Reference is made to FIG. 2. FIG. 2 is a schematic diagram illustrating a disability level automatic judgment method 200 according to some embodiments of the present disclosure. The embodiments of the present disclosure are not limited thereto.

It should be noted that, this disability level automatic judgment method 200 can be applied to systems with the same or similar structures as the disability level automatic judgment device 100 in FIG. 1. To make the description simple, the following will take FIG. 1 as an example to describe the operation method, but the embodiments of the present invention are not limited to the application of FIG. 1.

It should be noted that, in some embodiments, The disability level automatic judgment method 200 can also be implemented as a computer program and stored in a non-transitory computer readable medium, so that the computer, the electronic device, or the aforementioned processor 130 of the disability level automatic judgment device 100 as shown in FIG. 1 reads the recording medium and executes this operation method. The processor may consist of one or more chips. The non-transitory computer-readable recording media can be read-only memory, flash memory, floppy disks, hard disks, optical discs, flash drives, tapes, databases that can be accessed over the Internet, or non-transitory computer with the same function can read the recording medium those who are familiar with this technology can easily think of.

In addition, it should be understood that the operations of the disability level automatic judgment method 200 mentioned in this embodiment can be adjusted according to actual needs, or even simultaneously or at the same time, unless the order is specifically stated. Partially executed simultaneously.

Furthermore, in different embodiments, these operations may also be adaptively added, replaced, and/or omitted.

Reference is made to FIG. 2. The disability level automatic judgment method 200 includes the following operations.

In operation S210, the standard disability graph is stored. Reference is made to FIG. 1 at the same time. In some embodiments, the operation S210 can be executed by the memory 110 as shown in FIG. 1.

In some embodiments, the standard disability graph is created according to text information by the processor as illustrated in FIG. 1. In some embodiments, the text information includes information such as the disability impairment table and the definition of various functional impairment levels.

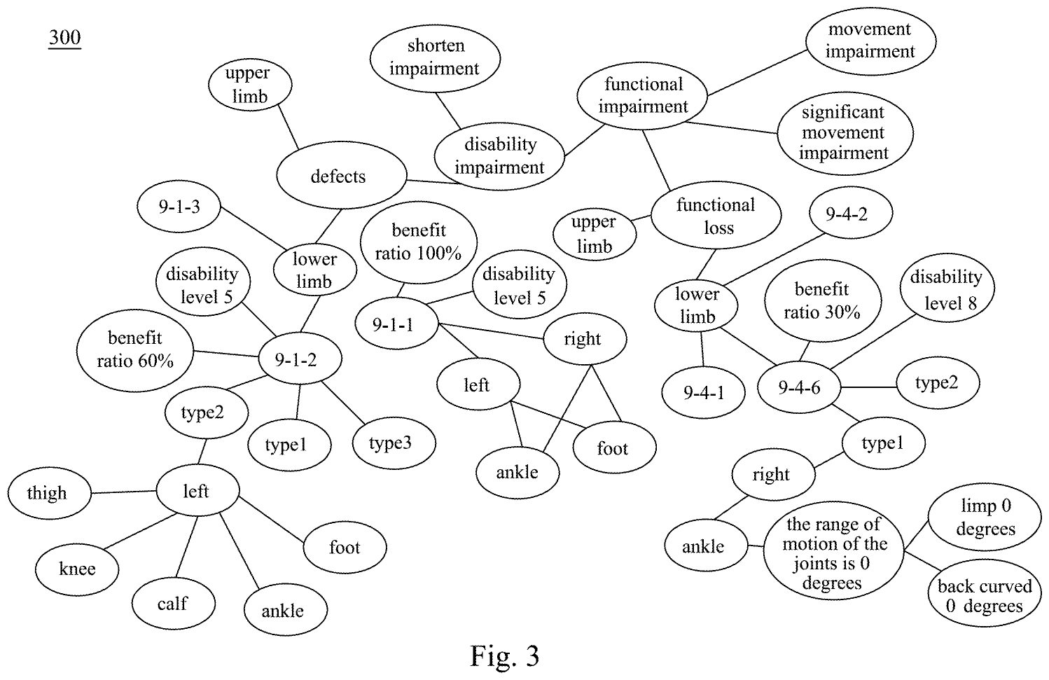

Reference is made to at the same time FIG. 3. FIG. 3 is a flowchart illustrating a standard disability graph 300 according to some embodiments of the present disclosure.

As illustrated in FIG. 3, the nodes in the standard disability graph 300 may include the body part, the diagnostic result, the disability level, the benefit ratio, etc., and the nodes are connected to each other.

Reference is made to again FIG. 2. In operation S230, the diagnosis information graph is created according to the diagnosis content. In some embodiments, the operation S230 can be executed by the processor 130 as shown in FIG. 1.

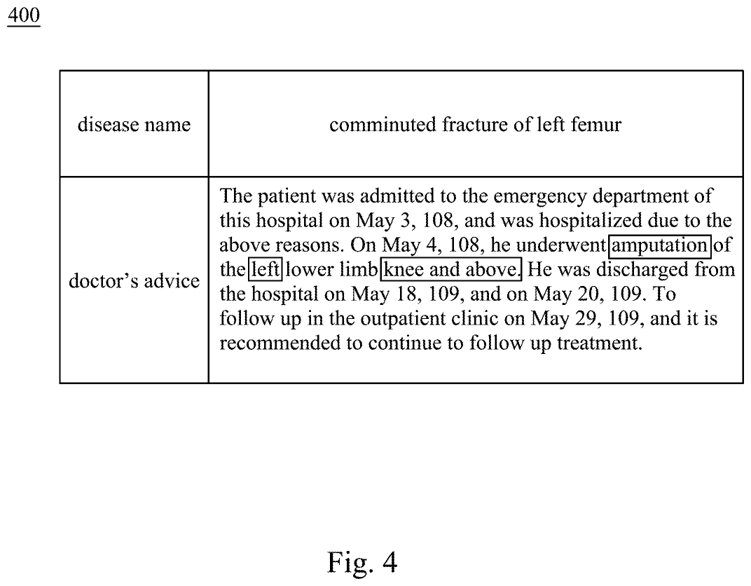

Reference is made to at the same time FIG. 4. FIG. 4 is a schematic diagram illustrating a diagnosis 400 according to some embodiments of the present disclosure.

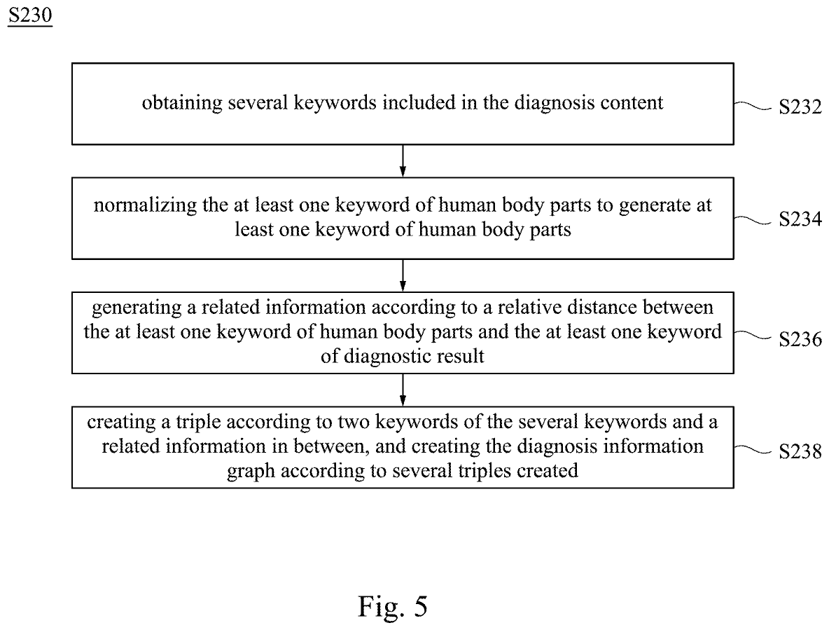

Reference is made to FIG. 5 at the same time. FIG. 5 is a flowchart illustrating the operation S230 illustrated in FIG. 2 according to some embodiments of the present disclosure.

In operation S232, several keywords included in the diagnosis content are obtained. In some embodiments, the operation S232 can be executed by the processor 130 as shown in FIG. 1.

In some embodiments, the keywords include the keyword of human body parts and the keyword of diagnostic results. For example, in the content of the diagnosis 400 in FIG. 4, the keyword of human body parts “left” and “knee above”, and the keyword of diagnostic result “amputation” are included.

In some other embodiments, the keywords can also include the position of the body (such as left right), the position of the body part (such as upper limb), the degree of damage or activity, the disability level, the impairment type, the benefit ratio, etc. The embodiments of the present disclosure are not limited to the keywords mentioned above.

In operation S234, the keyword of human body parts is normalized to generate a normalized keyword of human body parts. In some embodiments, the operation S234 can be executed by processor 130 as shown in FIG. 1. In some embodiments, the processor 130 generates the normalized keyword of human body parts according to the synonym comparison table of body positions and the graph of human body parts. For example, after the keyword of human body parts “knee above” is normalized, the normalized keyword of human body parts “thigh” is generated. In some embodiments, the normalized keyword of human body parts can further include thigh, knee, calf, ankle, foot, etc.

In operation S236, according to the relative distance between the keyword of human body parts and the keyword of diagnostic result, the related information is generated. In some embodiments, the operation S236 can be executed by processor 130 as shown in FIG. 1. In some embodiments, the related information can also include directional information, in which directional information is pointed from the keyword of diagnostic result to the keyword of human body parts. In some embodiments, the distance between keywords is based on the number of characters between keywords as the determination of the keyword relative distance.

For example, in the content of diagnosis 400 in FIG. 4, the relative distance between the keyword of human body parts “left” and the keyword of human body parts “above knee” is closer than the relative distances between the keyword of human body parts “left” and the other keywords, and relative distance between the keyword of human body parts “above knee” and the keyword of diagnostic result “amputation” is closer than the relative distances between the keyword of human body parts “above knee” and the other keywords. The processor 130 in FIG. 1 creates the related information according to the relative distance of the keyword of human body parts “left”, the keyword of human body parts “above knee” and the keyword of diagnostic result “amputation”. The related information includes the directional information. For example, the directional information points from the normalized keyword of human body parts “thigh” and the keyword of human body parts “left” to the keyword of diagnostic result “amputation”.

In operation S238, the triples are created according to the keywords and the related information in between, and the created diagnosis information graph is created according to the several triples created. In some embodiments, the operation S238 can be executed by the processor 130 as shown in FIG. 1. For example, the processor 130 creates the triple according to the related information between the keyword of human body parts “left”, the normalized keyword of human body parts “thigh” and the keyword of diagnostic result “amputation”, and the processor 130 creates the diagnosis information graph according to the triple.

It should be noted that, the content of the diagnosis 400 in FIG. 4 only includes one triple, but in some other embodiments, the content of diagnosis 400 may include several triples.

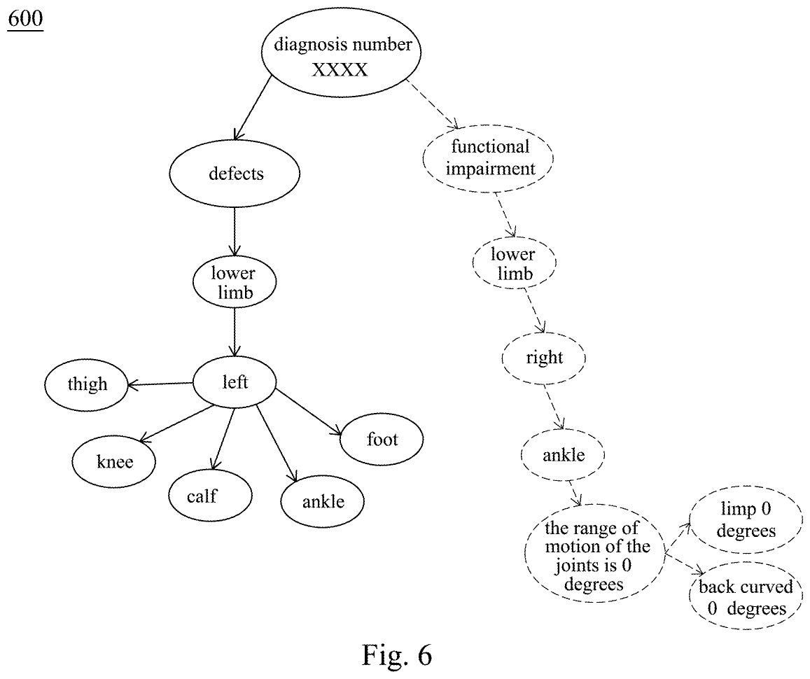

Reference is made to FIG. 6 at the same time. FIG. 6 is a schematic diagram illustrating a diagnosis information graph 600 according to some embodiments of the present disclosure. As shown in FIG. 6, in the diagnosis information graph 600, the keyword of diagnostic result “amputation” points to the normalized keyword of human body parts “thigh”, and then the normalized keyword of human body parts “thigh” points to the keyword of human body parts “left”.

In some embodiments, the processor 130 in FIG. 1 obtains several keywords included in the content of the diagnosis 400, and according to each two keywords of the several keywords and the related information in between, the triples are created, and then according to the created triples the diagnosis information graph is created. In some embodiments, the processor 130 selects a keyword of diagnostic result and a keyword of human body parts in the diagnosis 400, the number of words between the selected keyword of diagnostic result and the keyword of human body parts is used as the distance, so as to determine the relation. Thereby, the keyword of human body parts that is closest to the keyword of diagnostic result can be found, and the related information can be determined from the semantics of the sentences between these two keywords, and the triple is created.

In some embodiments, the keywords include the keyword of human body parts and the keyword of diagnostic result. The processor 130 further normalizes the keyword of human body parts to generate a normalized keyword of human body parts. In some embodiments, the processor 130 generates the at least one normalized keyword of human body parts according to a synonym comparison table of body positions and a graph of human body parts.

Reference is made to FIG. 2 again. In operation S250, the diagnosis information graph and the standard disability graph are compared, so as to determine the disability level. In some embodiments, operation S250 can be executed by processor 130 as shown in FIG. 1.

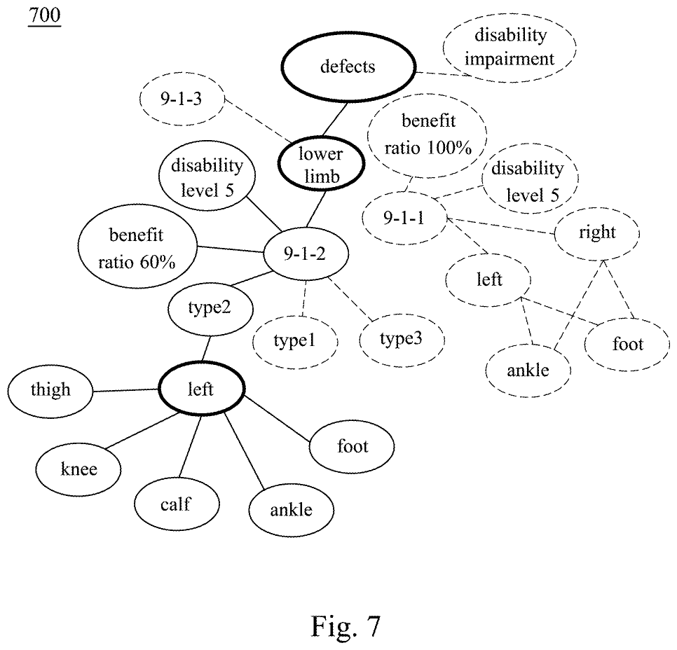

Reference is made to at the same time FIG. 7. FIG. 7 is a schematic diagram illustrating a comparison result 700 according to some embodiments of the present disclosure. In some embodiments, after the processor 130 compares the diagnosis information graph 600 in FIG. 6 with the standard disability graph 300 in FIG. 3, as shown in the comparison result 700, it is obtained that the closest disability level of the diagnosis information graph 600 to the standard disability graph 300 is disability level 5, 9-1-2, type 2.

Reference is made to FIG. 2 again. In operation S270, the judgment result is generated according to disability level. In some embodiments, the operation S270 can be executed by the processor 130 as shown in FIG. 1. In some embodiments, the judgment result is shown by the circuit for input and output 150 in FIG. 1.

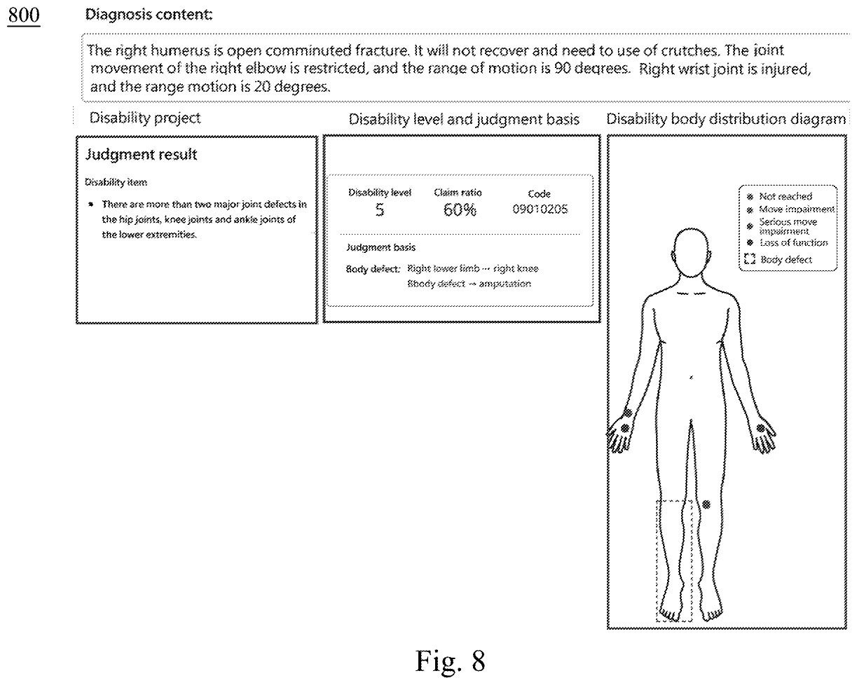

Reference is made to FIG. 8 at the same time. FIG. 8 is a schematic diagram illustrating a judgment result 800 according to some embodiments of the present disclosure. As illustrated in FIG. 8, there are three columns in the judgment result 800: the disability project, the disability level and the judgement basis, and the disability body distribution diagram.

In the column of the disability project, the keywords and the triples thereof obtained from the content of diagnosis 400 in FIG. 4 are displayed in a bar form.

In the disability level and the judgement basis, the determined disability level, the comparison similarity and other information are displayed.

In the disability body distribution diagram, the impairment body location and its corresponding impairment type determined by the content of diagnosis 400 in FIG. 4 are displayed.

In some embodiments, when both of the first disability level and the second disability level are determined for the same limb part, the judgment result is generated by the processor 130 according to the disability level of the first disability level and the second disability level in FIG. 1. For example, when the processor 130 determines several disability levels including the disability level 1 and the disability level 2 according to the lower limb of the content of the diagnosis 400, if the disability degree of the disability level 1 is more serious than the disability level 2, the processor 130 takes disability level 1 with the more severe disability as the determination result. The disability level can be judged according to the disability level table set by the government unit or insurance company, such as the labor insurance disability payment standard schedule, the disability impairment table, the disability degree table, the disability degree and the insurance payment table, etc. The text information of the standard disability graph 300 can also be created using the above disability level table and created by the technology of create diagnosis information graph, which will not be repeated here. In some embodiments, the above disability level table is a structured table with professional knowledge. To achieve higher accuracy, it can be created by domain experts according to the above disability level table and the knowledge graph software can be created.

In some embodiments, the processor 130 can be a server or other devices. In some embodiments, the processor 130 can be a server, a circuit, a central processing unit (CPU), a microprocessor (MCU), or other devices with the functions of storage, calculation, data reading, receiving signals or messages, and sending signals or messages.

In some embodiments, the memory 110 may be a device with functions of data storage or a device with similar functions. In some embodiments, the input/output circuit 170 may be a component with functions of signal output and signal input or similar functions.

According to the embodiment of the present disclosure, it is understood that the embodiment of the present disclosure is to provide a disability level automatic judgment device and a disability level automatic judgment method, according to diagnosis information the disability status and quickly can be analyzed and the most severe disability level judgment of each limb are provided. By using keyword normalization, the positions of the affected parts listed in the diagnosis are integrated, the repeated judgments of the same affected parts are avoided, and the accuracy of body part judgments is improved. Furthermore, according to the directional entity distance between keyword, the relational technology is created, the entity related information required for complete disability level judgment is obtained, which greatly improves the accuracy of diagnosis disability level judgment. Moreover, through the establishment of the graph, a quick correlation path comparison to find out the disability level that meets the requirements is performed, and the most severe disability level judgment result for each limb is provided. If several matching disability levels are found, the disability level with the most severe disability degree is used as the judgment result. In the presentation of the determination result, the disability part is directly presented in a graphic format, providing personnel to quickly confirm the result, and there is no need to look for disability determination related information in the diagnosis text one by one, and the correctness can be effectively verified.

In this document, the term “coupled” may also be termed as “electrically coupled”, and the term “connected” may be termed as “electrically connected”. “Coupled” and “connected” may also be used to indicate that two or more elements cooperate or interact with each other. It will be understood that, although the terms “first,” “second,” etc., may be used herein to describe various elements, these elements should not be limited by these terms. These terms are used to distinguish one element from another. For example, a first element could be termed a second element, and, similarly, a second element could be termed a first element, without departing from the scope of the embodiments. As used herein, the term “and/or” includes any and all combinations of one or more of the associated listed items.

In addition, the above illustrations include sequential demonstration operations, but the operations need not be performed in the order shown. The execution of the operations in a different order is within the scope of this disclosure. In the spirit and scope of the embodiments of the present disclosure, the operations may be increased, substituted, changed and/or omitted as the case may be.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A disability level automatic judgment device, comprising:
a processor, configured to create a diagnosis information graph according to a diagnosis content, and configured to compare the diagnosis information graph and a standard disability graph, so as to determine a first disability level, and to generate a judgment result according to the first disability level; and
a memory, coupled to the processor, and configured to store the standard disability graph.

2. The disability level automatic judgment device of claim 1, wherein the processor is further configured to create the standard disability graph according to a text information.

3. The disability level automatic judgment device of claim 1, wherein the processor is further configured to obtain a plurality of keywords comprised in the diagnosis content, and according to two keywords of the plurality of keywords and a related information in between to create a triple, and further to create the diagnosis information graph according to a plurality of triples created.

4. The disability level automatic judgment device of claim 3, wherein the plurality of keywords comprise at least one keyword of human body parts and at least one keyword of diagnostic result.

5. The disability level automatic judgment device of claim 4, wherein the processor is further configured to normalize the at least one keyword of human body parts, to generate at least one normalized keyword of human body parts.

6. The disability level automatic judgment device of claim 5, wherein the processor is further configured to generate the at least one normalized keyword of human body parts according to a synonym comparison table of body positions and a graph of human body parts.

7. The disability level automatic judgment device of claim 4, wherein the processor is further configured to generate the triple according to a relative distance between the at least one keyword of human body parts and the at least one keyword of diagnostic result from the plurality of keywords.

8. The disability level automatic judgment device of claim 5, wherein the related information further comprises a directional information, wherein the directional information points from the at least one keyword of diagnostic result to the at least one keyword of human body parts.

9. The disability level automatic judgment device of claim 1, wherein when the processor compares the diagnosis information graph and the standard disability graph and when the first disability level and a second disability level are determined for a same body part, the processor is further configured to generate the judgment result according to a disability level of the first disability level and the second disability level.

10. The disability level automatic judgment device of claim 1, further comprising:
a circuit for input and output, configured to display the judgment result, wherein the judgment result comprises a disability body distribution diagram, so as to mark an impairment body location and an impairment type in correspondence determined according to the diagnosis content.

11. A disability level automatic judgment method, comprising:
storing a standard disability graph by a memory;
creating a diagnosis information graph according to a diagnosis content by a processor;
comparing the diagnosis information graph and the standard disability graph, so as to determine a first disability level by the processor; and
generating a judgment result according to the first disability level by the processor.

12. The disability level automatic judgment method of claim 11, further comprising:
creating the standard disability graph according to a text information by the processor.

13. The disability level automatic judgment method of claim 11, further comprising:
obtaining a plurality of keywords comprised in the diagnosis content, creating a triple according to two keywords of the plurality of keywords and a related information in between, and creating the diagnosis information graph according to a plurality of triples created by the processor.

14. The disability level automatic judgment method of claim 13, wherein the plurality of keywords comprise at least one keyword of human body parts and at least one keyword of diagnostic result.

15. The disability level automatic judgment method of claim 14, further comprising:
normalizing the at least one keyword of human body parts to generate at least one normalized keyword of human body parts by the processor.

16. The disability level automatic judgment method of claim 15, further comprising:
generating the at least one normalized keyword of human body parts according to a synonym comparison table of body positions and a graph of human body parts by the processor.

17. The disability level automatic judgment method of claim 14, further comprising:
generating the triple according to a relative distance between the at least one keyword of human body parts and the at least one keyword of diagnostic result from the plurality of keywords by the processor.

18. The disability level automatic judgment method of claim 15, wherein the related information further comprises a directional information, wherein the directional information points from the at least one keyword of diagnostic result to the at least one keyword of human body parts.

19. The disability level automatic judgment method of claim 11, further comprising:
generating the judgment result by the processor according to a disability level of the first disability level and a second disability level when the processor compares the diagnosis information graph and the standard disability graph and when the first disability level and the second disability level are determined for a same body part.

20. The disability level automatic judgment method of claim 11, further comprising:
displaying the judgment result by a circuit for input and output, wherein the judgment result comprises a disability body distribution diagram, so as to mark an impairment body location and an impairment type in correspondence determined according to the diagnosis content.
